(19) United States
(12) Patent Application Publication
(10) Pub. No.: US 2023/0039410 A1
(43) Pub. Date: Feb. 9, 2023

(54) SEMICONDUCTOR ULTRAVIOLET LIGHT EMITTING DEVICE PACKAGE

(71) Applicant: SAMSUNG ELECTRONICS CO., LTD., Suwon-si (KR)

(72) Inventors: Junghun Lee, Suwon-si (KR); Kyoungjun Kim, Yongin-si (KR); Sun Kim, Hwaseong-si (KR); Seolyoung Choi, Yongin-si (KR)

(73) Assignee: SAMSUNG ELECTRONICS CO., LTD., Suwon-si (KR)

(21) Appl. No.: 17/730,336

(22) Filed: Apr. 27, 2022

(30) Foreign Application Priority Data
Aug. 3, 2021 (KR) 10-2021-0102118

(51) Int. Cl. H01L 33/60; H01L 33/48; A61L 2/10

(52) U.S. Cl. CPC H01L 33/60; A61L 2/10; H01L 33/483; H01L 33/32

(57) ABSTRACT

A semiconductor ultraviolet light emitting device package is provided. The semiconductor ultraviolet light emitting device package includes: a semiconductor ultraviolet light emitting device mounted on the first surface of the package substrate and configured to emit deep ultraviolet light including a wavelength in a range of 250 nm to 285 nm; a reflector disposed on the first surface of the package substrate to surround the semiconductor ultraviolet light emitting device, and including an inclined sidewall that defines an opening of the reflector, the semiconductor ultraviolet light emitting device disposed within the opening; and a light transmitting cover including a lower surface covering the opening and an upper surface opposite to the lower surface, wherein an antireflective layer is disposed on at least one from among the lower surface and the upper surface.

### CROSS-REFERENCE TO THE RELATED APPLICATION

This application claims benefit of priority to Korean Patent Application No. 10-2021-0102118 filed on Aug. 3, 2021, in the Korean Intellectual Property Office, the disclosure of which is incorporated herein by reference in its entirety.

### BACKGROUND

Embodiments of the present disclosure relate to a semiconductor ultraviolet light emitting device package.

Recently, ultraviolet light sources have been used for various purposes such as sterilization and disinfection devices, UV curing devices, and the like. As such a UV light source, an eco-friendly and high-efficiency UV light emitting diode (UV LED) is attracting attention.

Sterilization power of ultraviolet light emitted from an ultraviolet light emitting diode is affected in a dosage amount, corresponding to the energy density. As an orientation angle of ultraviolet light increases, since the dosage decreases and the sterilization power decreases, a technique in which an orientation angle of ultraviolet emitted from the ultraviolet light emitting diode may be narrowed is required.

### SUMMARY

An aspect of the present disclosure is to provide a semiconductor ultraviolet light emitting device package emitting ultraviolet light having a narrow orientation angle.

According to embodiments, a semiconductor ultraviolet light emitting device package is provided. The semiconductor ultraviolet light emitting device package includes: a package substrate including a first surface and a second surface, opposite to the first surface; a semiconductor ultraviolet light emitting device mounted on the first surface of the package substrate and configured to emit deep ultraviolet light including a wavelength in a range of 250 nm to 285 nm; a reflector disposed on the first surface of the package substrate to surround the semiconductor ultraviolet light emitting device, and including an inclined sidewall that defines an opening of the reflector, the semiconductor ultraviolet light emitting device disposed within the opening; and a light transmitting cover including a lower surface covering the opening and an upper surface opposite to the lower surface, wherein an antireflective layer is disposed on at least one from among the lower surface and the upper surface, wherein the inclined sidewall has an inclination angle greater than 55° and less than 75° with respect to the first surface, wherein a distance between an upper surface of the semiconductor ultraviolet light emitting device and the lower surface of the light transmitting cover is 100 µm to 2000 µm, and wherein a refractive index of the antireflective layer is in a range of 1.3 to 2.5.

According to embodiments, a semiconductor ultraviolet light emitting device package is provided. The semiconductor ultraviolet light emitting device package includes: a body having a cavity; a semiconductor ultraviolet light emitting device mounted on a surface of the body that defines a bottom of the cavity; a light transmitting cover including a light transmitting substrate covering the cavity; and an antireflective layer disposed on at least one from among an upper surface and a lower surface of the light transmitting substrate, wherein the body includes a sidewall that defines sides of the cavity and has an inclination angle that is greater than 55° and less than 75° with respect to the surface of the body that defines the bottom of the cavity, wherein a distance between an upper surface of the semiconductor ultraviolet light emitting device and a lower surface of the light transmitting cover is 100 µm to 2000 µm, and wherein a refractive index of the antireflective layer is in a range of 1.3 to 2.5.

According to embodiments, a semiconductor ultraviolet light emitting device package is provided. The semiconductor ultraviolet light emitting device package includes: a package substrate; a reflector disposed on the package substrate and including an inclined sidewall that defines an opening of the reflector; a semiconductor ultraviolet light emitting device mounted on the package substrate and disposed in the opening; and a light transmitting cover including a lower surface covering the opening and an upper surface opposite to the lower surface, wherein an antireflective layer is disposed on at least one from among the lower surface and the upper surface, wherein the inclined sidewall has an inclination angle that is greater than 55° and less than 75° with respect to an upper surface of the package substrate, and wherein a refractive index of the antireflective layer is in a range of 1.3 to 2.5.

### BRIEF DESCRIPTION OF DRAWINGS

The above and other aspects, features, and advantages of embodiments of the present disclosure will be more clearly understood from the following detailed description, taken in conjunction with the accompanying drawings, in which:

FIG. 1 is a perspective view of a semiconductor ultraviolet light emitting device package according to an example embodiment of the present disclosure;

FIG. 2 is a cross-sectional view of the semiconductor ultraviolet light emitting device package of FIG. 1 taken along line I-I';

FIG. 3 is a diagram schematically illustrating that light emitted from the semiconductor ultraviolet light emitting device of FIG. 2 passes through an antireflective layer;

FIG. 4A is a cross-sectional view illustrating a semiconductor ultraviolet light emitting device for employment in the semiconductor ultraviolet light emitting device package of FIG. 1;

FIG. 4B is a cross-sectional view illustrating a semiconductor ultraviolet light emitting device for employment in the semiconductor ultraviolet light emitting device package of FIG. 1;

FIG. 5 is a graph illustrating transmittance and a maximum transmittance angle according to types of antireflective layers in semiconductor ultraviolet light emitting device packages according to Examples and a Comparative Example;

FIG. 6 is a graph illustrating a relationship between an incident angle of ultraviolet light incident on antireflective layers formed on each semiconductor ultraviolet light emitting device package and transmittance in the semiconductor ultraviolet light emitting device packages of Examples and a Comparative Example;

FIG. 7 is a perspective view of a semiconductor ultraviolet light emitting device package of an example embodiment of the present disclosure;

FIG. 8 is a cross-sectional view of the semiconductor ultraviolet light emitting device package of FIG. 7 taken along line II-II';

FIG. 9 is a first cross-sectional view schematically illustrating a manufacturing process of the semiconductor ultraviolet light emitting device package of FIGS. 1 and 2;

FIG. 10 is a second cross-sectional view schematically illustrating the manufacturing process of the semiconductor ultraviolet light emitting device package of FIGS. 1 and 2;

FIG. 11 is a third cross-sectional view schematically illustrating the manufacturing process of the semiconductor ultraviolet light emitting device package of FIGS. 1 and 2; and

FIG. 12 is a fourth cross-sectional view schematically illustrating the manufacturing process of the semiconductor ultraviolet light emitting device package of FIGS. 1 and 2.

### DETAILED DESCRIPTION

Hereinafter, example embodiments of the present disclosure will be described with reference to the accompanying drawings.

It will be understood that when an element or layer is referred to as being “over,” “above,” “on,” “below,” “under,” “beneath,” “connected to”or “coupled to” another element or layer, it can be directly over, above, on, below, under, beneath, connected or coupled to the other element or layer or intervening elements or layers may be present. In contrast, when an element is referred to as being “directly over,” “directly above,” “directly on,” “directly below,” “directly under,” “directly beneath,” “directly connected to” or “directly coupled to” another element or layer, there are no intervening elements or layers present.

As used herein, expressions such as “at least one of (among),” when preceding a list of elements, modify the entire list of elements and do not modify the individual elements of the list. For example, the expression, “at least one of (among) a, b, and c,” should be understood as including only a, only b, only c, both a and b, both a and c, both b and c, or all of a, b, and c.

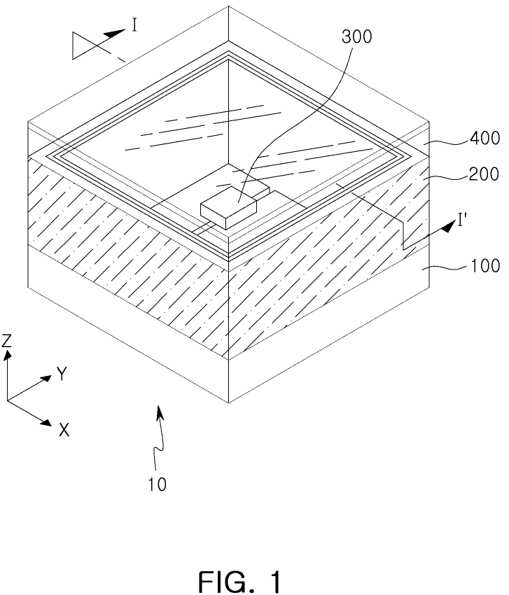

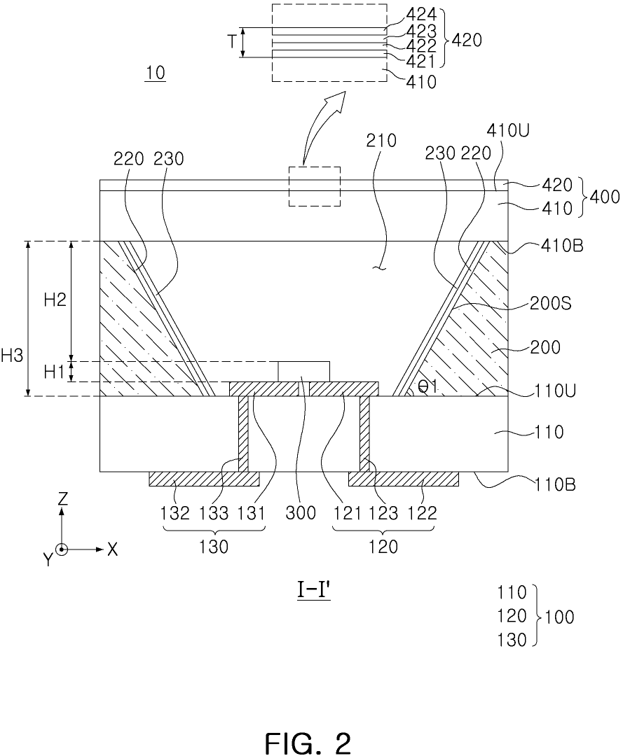

Referring to FIGS. 1 and 2, a semiconductor ultraviolet light emitting device package 10 according to an example embodiment of the present disclosure will be described. FIG. 1 is a perspective view of a semiconductor UV light emitting device package according an example embodiment of the present disclosure, and FIG. 2 is a cross-sectional view of the semiconductor UV light emitting device package of FIG. 1 taken along line I-I'. In this specification, terms such as ‘on’, ‘upper portion’, ‘upper surface’, ‘under’, ‘lower portion’, 'lower surface’, ‘side surface’ are based on the drawings, and in fact, may vary depending on a direction in which devices or packages are disposed.

The light emitting device package according to an example embodiment of the present disclosure may include a package substrate 100, a semiconductor ultraviolet light emitting device 300, a reflector 200, and a light transmitting cover 400.

The package substrate 100 may include a package body 110, a first electrode structure 120, and a second electrode structure 130.

The package body 110 may have a substrate shape having an upper surface 110U and a lower surface 110B, and may provide a mounting region in which the semiconductor ultraviolet light emitting device 300 is mounted. The package substrate 100 may be formed of an organic resin material containing epoxy, triazine, silicon, polyimide, or the like, and other organic resin materials, but in order to improve heat dissipation characteristics and luminance efficiency, the package substrate 100 may be formed of a ceramic material having characteristics such as high heat resistance, excellent thermal conductivity, high reflection efficiency, or the like, for example, a material such as Al₂O₃, or AlN. However, the material of the package body 110 is not limited thereto, and various materials of the package body 110 may be used in consideration of the heat dissipation characteristics and electrical connection relationship of the semiconductor ultraviolet light emitting device package 10. In addition to the above-described ceramic substrate, a printed circuit board, a lead frame, or the like, may also be used as the package body 110 of the present example embodiment.

The first electrode structure 120 and the second electrode structure 130 may be disposed on the package body 110. The first electrode structure 120 and the second electrode structure may include a first upper electrode 121, a second upper electrode 131, a first lower electrode 122, a second lower electrode 132, a first through electrode 123, and a second through electrode 133. The first upper electrode 121 and the second upper electrode 131 may be disposed on the upper surface 110U of the package body 110. The first lower electrode 122 and the second lower electrode 132 may be disposed on the lower surface 110B of the package body 110. The first through electrode 123 and the second through electrode 133 may penetrate through the upper surface 110U and the lower surface 110B of the package body 110 to electrically connect the first upper electrode 121 and the second upper electrode 131 and the first lower electrode 122 and the second lower electrode 132 to each other, respectively. However, the shapes of the first electrode structure 120 and the second electrode structure are not limited to the illustrated ones, and may be modified into various shapes.

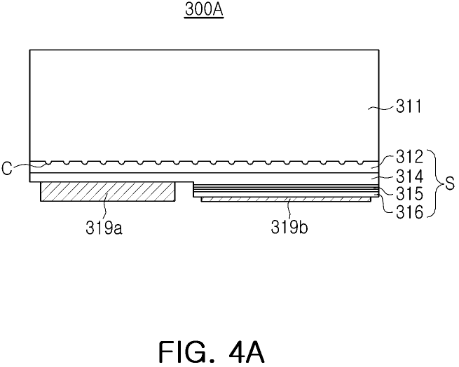

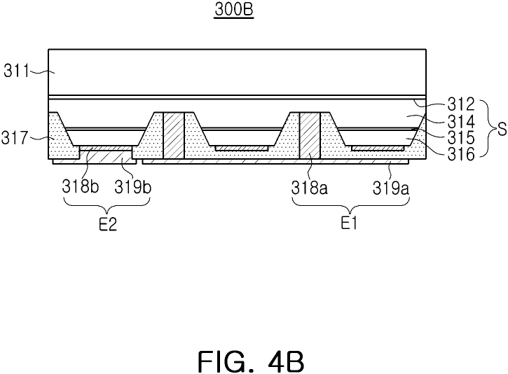

The semiconductor ultraviolet light emitting device 300 may be mounted on the package substrate 100. In an example embodiment, the semiconductor ultraviolet light emitting device 300 may be mounted on the first upper electrode 121 and the second upper electrode 131 of the package substrate 100. The semiconductor ultraviolet light emitting device 300 for employment in the semiconductor ultraviolet light emitting device package 10 will be described with reference to FIGS. 4A and 4B.

The semiconductor ultraviolet light emitting device 300 according to an example embodiment may output light in an ultraviolet wavelength band. For example, the semiconductor ultraviolet light emitting device 300 may output light (UV-A) in a near-ultraviolet wavelength band, output light (UV-B) in a far-ultraviolet wavelength band, and output light (UV-C) in a deep-ultraviolet wavelength band. A wavelength range of the output light emitted from the semiconductor ultraviolet light emitting device 300 may be determined by an Al composition ratio of a light emitting structure S included in the semiconductor ultraviolet light emitting device 300.. For example, the light (UV-A) in the near-ultraviolet wavelength band may have a wavelength band in a range of 320 nm to 420 nm as a central wavelength, and the light (UV-B) in the far-ultraviolet wavelength band may have a wavelength band in a range of 285 nm to 320 nm as a central wavelength, and the light (UV-C) in the deep-ultraviolet wavelength band may have a wavelength band in a range of 100 nm to 285 nm as a central wavelength. The semiconductor ultraviolet light emitting device 300 according to an example embodiment may output light in a deep-ultraviolet wavelength band having a wavelength band in a range of 250 nm to 285 nm as a central wavelength.

The semiconductor ultraviolet light emitting device 300 employed in an example embodiment may have a flip-chip structure in which a surface from which light is emitted and a surface on which electrodes are disposed are opposite to each other. However, embodiments of the present disclosure are not limited thereto, and various types of semiconductor ultraviolet light emitting devices may be employed. FIGS. 4A and 4B are cross-sectional views illustrating various semiconductor ultraviolet light emitting devices employable in the semiconductor ultraviolet light emitting device package 10.

Referring to FIG. 4A, the semiconductor ultraviolet light emitting device 300A may include a substrate 311, and the light emitting structure S in which a first conductivity-type semiconductor layer 314, an active layer 315, and a second conductivity-type semiconductor layer 316 are sequentially disposed on the substrate 311. A buffer layer 312 may be disposed between the substrate 311 and the first conductivity-type semiconductor layer 314.

The substrate 311 may be an insulating substrate such as sapphire. However, embodiments of the present disclosure are not limited thereto, and the substrate 311 may be a conductive or semiconductor substrate in addition to an insulating substrate. For example, the substrate 311 may be SiC, Si, MgAl₂O₄, MgO, LiA1O₂, LiGa_{O2}, or GaN in addition to sapphire. Unevenness C may be formed on an upper surface of the substrate 311. The unevenness C may improve quality of the grown single crystal while improving light extraction efficiency.

The first conductivity-type semiconductor layer 314 may be an n-type nitride semiconductor represented by Alₓ₁Ga₁₋ₓ₁N (0<x1≤1), and an n-type impurity may be Si. For example, the first conductivity-type semiconductor layer 314 may include n-type AlGaN. A second conductivity-type semiconductor layer 316 may be a p-type nitride semiconductor expressed by Alₓ₂Ga₁₋ₓ₂N (0<x2≤1), and the p-type impurity may be Mg. For example, the second conductivity-type semiconductor layer 316 may include p-type AlGaN.

In an example, an Al composition ratio (x1) of the first conductivity-type semiconductor layer 314 may be in a range of 0.55 to 0.70, and further may be in a range of 0.60 to 0.65. Similarly, an Al composition ratio (x2) of the second conductivity-type semiconductor layer 316 may be in a range of 0.55 to 0.70, and further may be in a range of 0.60 to 0.65.

The active layer 315 employed in this example embodiment may have a quantum well formed of Alₓ₃Ga₁₋ₓ₃N(0<x3<1). The active layer 315 may be a single-quantum well (SQW) structure having one quantum well, but is not limited thereto, and the active layer 315 may have a multi-quantum well (MQW) structure in which a plurality of well structures formed of AlₓₐGa₁₋ ₓₐN(0<xa<1) and a plurality of multi-quantum barrier structures formed of Al_{xb}Ga_{1-xb}N(xa<xb<1) are alternately stacked.

The quantum well of the active layer 315 has a band gap determining a wavelength of ultraviolet light, and the active layer 315 employed in this embodiment may be configured to emit deep ultraviolet light having a wavelength of 250 to 285 nm. The first conductivity-type semiconductor layer 314 and the second conductivity-type semiconductor layer 316 may have a band gap greater than that of the quantum well so that ultraviolet light generated from the active layer 315 is not absorbed. For example, an Al composition ratio (x3 or xa) of the quantum well may be smaller than the Al composition ratio (x1 and x2) of the first conductivity-type semiconductor layer 314 and the second conductivity-type semiconductor layer 316. In one example, the Al composition ratio (x3 or xa) of the quantum well may be in a range of 0.35 to 0.5.

A first electrode pad 319a and a second electrode pad 319b may be respectively disposed in a mesa-etched region of the first conductivity-type semiconductor layer 314 and the second conductivity-type semiconductor layer 316 so as to be positioned to face the same direction. The first electrode pad 319a is not limited thereto, but may include a material such as Ag, Ni, Al, Cr, Rh, Pd, Ir, Ru, Mg, Zn, Pt, Au, and the like, and may be employed as a structure of a single layer or two or more layers. According to embodiments, the second electrode pad 319b may be a transparent electrode such as a transparent conductive oxide or a transparent conductive nitride, or may include graphene. The second electrode pad 319b may include at least one of A1, Au, Cr, Ni, Ti, and Sn. The first electrode pad 319a and the second electrode pad 319b may be connected to the first electrode structure 120 and the second electrode structure of FIG. 1, respectively.

FIG. 4B is a side cross-sectional view illustrating an example of another type of semiconductor ultraviolet light emitting device that may be employed in this example embodiment.

Referring to FIG. 4B, a semiconductor ultraviolet light emitting device 300B may include the substrate 311, and the light emitting structure S disposed on the substrate 311, similarly to the previous embodiment. The light emitting structure S may include a buffer layer 312, a first conductivity-type semiconductor layer 314, an active layer 315, and a second conductivity-type semiconductor layer 316.

The semiconductor ultraviolet light emitting device 300B may include a first electrode pad structure E1 and a second electrode pad structure E2 respectively connected to the first conductivity-type semiconductor layer 314 and the second conductivity-type semiconductor layer 316. The first electrode pad structure E1 may include a connection electrode 318a such as a conductive via penetrating through the second conductivity-type semiconductor layer 316 and the active layer 315 and connected to the first conductivity-type semiconductor layer 314, and a first electrode pad 319a connected to the connection electrode 318a. The connection electrode 318a may be surrounded by an insulating portion 317 and may be electrically separated from the active layer 315 and the second conductivity-type semiconductor layer 316. The connection electrode 318a may be disposed in a region from which the light emitting structure S is etched. The number, shape, pitch, or contact area of the connection electrode 318a with the first conductivity-type semiconductor layer 314 may be appropriately designed so that a contact resistance is lowered. In addition, the connection electrode 318a may be arranged on the light emitting structure S to form rows and columns, thereby improving current flow. The second electrode pad structure E2 may include an ohmic contact layer 318b and a second electrode pad 319b on the second conductivity-type semiconductor layer 316.

The connection electrode 318a and the ohmic contact layer 318b may respectively include a single layer or multilayer structure with the first conductivity-type semiconductor layer 314 and the second conductivity-type semiconductor layer 316 formed of a conductive material having an ohmic characteristic, and for example, may include a material such as Ag, Al, Ni, Cr, and a transparent conductive oxide (TCO).

The first electrode pad 319a and the second electrode pad 319b may be respectively connected to the connection electrode 318a and the ohmic contact layer 318b to function as an external terminal of the semiconductor UV light emitting device 300B. For example, the first electrode pad 319a and the second electrode pad 319b may include Au, Ag, Al, Ti, W, Cu, Sn, Ni, Pt, Cr, NiSn, TiW, AuSn, or a eutectic metal thereof. The first electrode pad structure E1 and the second electrode pad structure E2 may be disposed in the same direction. The first electrode pad 319a and the second electrode pad 319b may be connected to the first electrode structure 120 and the second electrode structure of FIG. 1, respectively.

Referring back to FIG. 2, a reflector 200 may be disposed on the upper surface 110U of the package body 110. An opening 210 in which the semiconductor ultraviolet light emitting device 300 is accommodated may be formed in a center of the reflector 200. A height H3 of the opening 210 may be greater than the height H1 of the semiconductor ultraviolet light emitting device 300 to be sufficient to accommodate the semiconductor ultraviolet light emitting device 300. In an example embodiment, the height H3 of the opening 210 may have a height of 300 µm to 2200 µm with respect to the upper surface 110U of the package substrate 100. For example, the height H3 of the opening 210 may have a height of 300 µm to 2200 µm with respect to the upper surface 110U of the package substrate 100. When the height H1 of the semiconductor ultraviolet light emitting device 300 is 200 µm, an upper surface of the semiconductor ultraviolet light emitting device 300 and a lower surface 410B of the light transmitting cover 400 have a distance H2 of 100 µm to 2000 µm therebetween. As the height H3 of the opening 210 increases, an orientation angle of light emitted from the semiconductor UV light emitting device 300 tends to decrease. However, when the height H3 of the opening 210 becomes excessively large, since the height of the semiconductor ultraviolet light emitting device package 10 is unnecessarily increased, utility of the semiconductor ultraviolet light emitting device package 10 may be limited.

A sidewall 200S of the opening 210 may surround a side surface of the semiconductor ultraviolet light emitting device 300 and may have a constant inclination angle θθ01. The opening 210 of the reflector 200 may have a structure in which a width is increased toward an upper portion thereof, and a cross section thereof may have a shape close to a rectangle. To this end, the sidewall 200S of the opening 210 may be formed as a flat inclined surface inclined upwardly from the upper surface 110U of the package body 110 toward the lower surface 410B of the light transmitting cover 400. The sidewall 200S may be disposed to have a predetermined inclination angle θ1. The inclination angle θ1 may be, for example, in a range of 45° to 75°, for example, in a range exceeding 55° and less than 75°. According to the inclination angle θ1 formed by the sidewall 200S of the reflector 200, an orientation angle of ultraviolet light emitted from the semiconductor ultraviolet light emitting device 300 may be adjusted. In addition, according to the inclination angle θ1 formed by the sidewall 200S of the reflector 200, a transmittance of the ultraviolet light passing through the light transmitting cover 400 may be adjusted. This will be described later.

The reflector 200 may be formed of a metal having high reflectivity, such as aluminum, in order to maintain high surface reflectivity. When the reflector 200 is formed of aluminum, a surface of the reflector 200 may be naturally oxidized to form a passivation film (AlₓO_{y}) having a thickness of about 10 nm. Such a passivation film may prevent the surface of aluminum from being further oxidized, but in a humid environment, an effect of preventing the surface of aluminum from being oxidized may be lowered. When the surface of the reflective layer 220 is oxidized, reflectance of the reflector 200 may be rapidly reduced. To prevent this, a protective layer 230 for preventing a surface of the reflector 200 from being oxidized may be formed on the surface of the reflector 200. The protective layer 230 may be formed of any one of SiO₂, Al₂O₃, AlN, and Si₃N₄, or a combination thereof. A thickness of the protective layer 230 may be in a range of 5 nm to about 50 nm.

In addition, the reflector 200 may be formed by forming a body of ceramic or silicon having relatively low reflectivity, and covering the sidewall 200S with a reflective layer 220 formed of a material having high reflectivity such as aluminum. A protective layer 230 for preventing the reflective layer 220 from being oxidized may be formed on a surface of the reflective layer 220.

The light transmitting cover 400 may be disposed on an upper surface of the reflector 200 to cover the opening 210 of the reflector 200. The light transmitting cover 400 may cover the opening 210 to block the semiconductor UV light emitting device 300 disposed on a bottom surface of the opening 210 from external moisture. The light transmitting cover 400 may cover the opening 210 to form a space isolated from the outside. This space may be filled with air, but is not limited thereto. According to example embodiments, the space may be in a vacuum state. In addition, the space may be filled with an inert gas having low chemical reactivity, such as helium (He), neon (Ne), Argon, or Xenon.

The light transmitting cover 400 may be attached to an upper surface of the reflector 200 by an adhesive layer. The adhesive layer may be formed of a material such as a silicone resin, an epoxy resin, an acrylic resin, a metal layer, and water glass or silicone. However, according to example embodiments, the adhesive layer may be omitted, and the light transmitting cover 400 may be attached to the upper surface of the reflector 200 by methods such as anodic bonding, fusion bonding, eutectic bonding, welding, or the like.

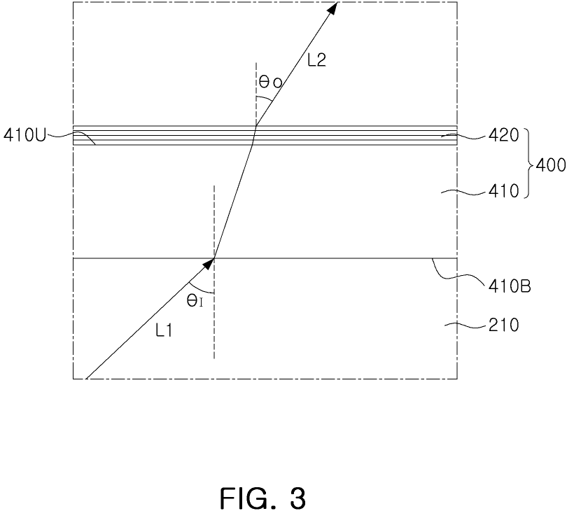

Referring to FIGS. 2 and 3, the light transmitting cover 400 may include a light transmitting substrate 410 and an antireflective layer 420.

The light transmitting substrate 410 may be formed in a substrate shape having a uniform thickness. The light transmitting substrate 410 may be formed of one of a material such as soft glass, fused silica, and fused quartz. In addition, the light transmitting substrate 410 may be formed of low-temperature sintered glass obtained by sintering a glass frit at a low temperature. As described above, since the light transmitting substrate 410 is formed of glass or quartz and a material that is not easily discolored by UV light, discoloration of the light transmitting cover 400 by UV light can be prevented. Accordingly, absorption of ultraviolet light emitted from the semiconductor ultraviolet light emitting device 300 by the light transmitting cover 400 may be minimized.

An antireflective layer 420 may be formed on at least one of an upper surface 410U and a lower surface 410B of the light transmitting substrate 410. According to an example embodiment, the antireflective layer 420 may be formed only on the upper surface 410U of the light transmitting substrate 410, and may also be formed on both the upper surface 410U and the lower surface 410B.

The antireflective layer 420 may be an antireflection coating layer.

A refractive index of the antireflective layer 420 may be selected according to a refractive index of the light transmitting substrate 410. For example, when the refractive index of the light transmitting substrate 410 is 1.45 to 1.55, the antireflective layer 420 may be formed of a material having a refractive index in a range of 1.3 to 2.5 or a combination of these materials.

The antireflective layer 420 may destructively interfere with a wavelength reflected from an interface between the light transmitting substrate 410 and the antireflective layer 420 (a lower surface of the antireflective layer 420) and an interface between the antireflective layer 420 and an air of the opening 210 (an upper surface of the antireflective layer 420) to reduce reflectivity and improve transmittance. Accordingly, the antireflective layer 420 may minimize light reflected from the interface to increase the transmittance of the ultraviolet light emitted from the semiconductor ultraviolet light emitting device 300.

The antireflective layer 420 may be formed of only an inorganic material without including an organic material such as a carbon compound. For example, the antireflective layer 420 may include at least one of MgF₂, SiO₂, Al₂O₃, and HfO₂.

The antireflective layer 420 may be formed as a single-layer structure, but may also be formed as a multilayer structure according to example embodiments. When the antireflective layer 420 is formed as a multilayer structure, the antireflective layer may have a structure in which two or more refractive layers (e.g. layers 421, 422, 423, and 424) having different refractive indices are stacked. For example, the antireflective layer 420 may have a structure in which a first refractive layer having a first refractive index and a second refractive layer having a second refractive index higher than the first refractive index are alternately stacked. In this case, the refractive layers may be stacked once, but may be repeatedly stacked twice or more.

When the semiconductor ultraviolet light emitting device 300 emits light having a wavelength band of 100 nm to 285 nm as a center wavelength, a thickness T of the antireflective layer 420 may be 25 nm to 400 nm.

Referring to FIG. 3, among the ultraviolet light emitted from the semiconductor ultraviolet light emitting device 300, incident light L1 incident on a lower surface 410B of the light transmitting cover 400 at a first incident angle θ1 may be refracted by a difference in refractive index between air filled in the opening 210 and the light transmitting substrate 410, and may be refracted on an upper surface 410U of the light transmitting substrate 410 by a difference in refractive index between the light transmitting substrate 410 and the antirefractive layer 420. Exit light L2 emitted through the antireflective layer 420 may be emitted at an exit angle θO smaller than the incidence angle θI. In addition, total reflection within the light transmitting substrate 410 may be minimized by the antireflective layer 420.

In addition, the antireflective layer 420 may be formed so that the ultraviolet light emitted from the semiconductor ultraviolet light emitting device 300 has transmittance of 95% or more with respect to the light transmitting cover 400. In addition, the antireflective layer 420 may be formed such that a maximum transmission angle of the ultraviolet light passing throughthe light transmitting cover 400 is 55° or more. The maximum transmission angle may be defined as an incident angle incidence when the transmittance is 90%. As the maximum transmission angle increases, a dosage of light irradiated to a front of the semiconductor UV light emitting device package 10 increases, and a direction of light irradiation also faces forward. Therefore, it can be understood that as the maximum transmission angle increases, the irradiated light is concentrated on a central axis of the light, and thus an irradiation angle of the light becomes narrower. For example, when the maximum transmission angle of light exceeds 55°, it may be irradiated with an orientation angle of less than 100°.

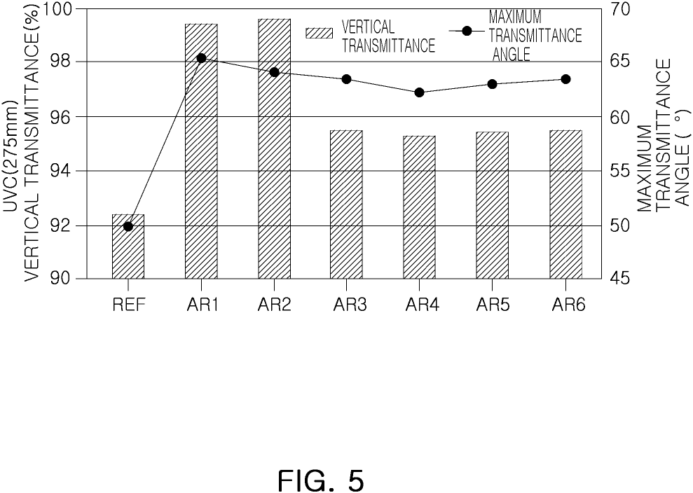

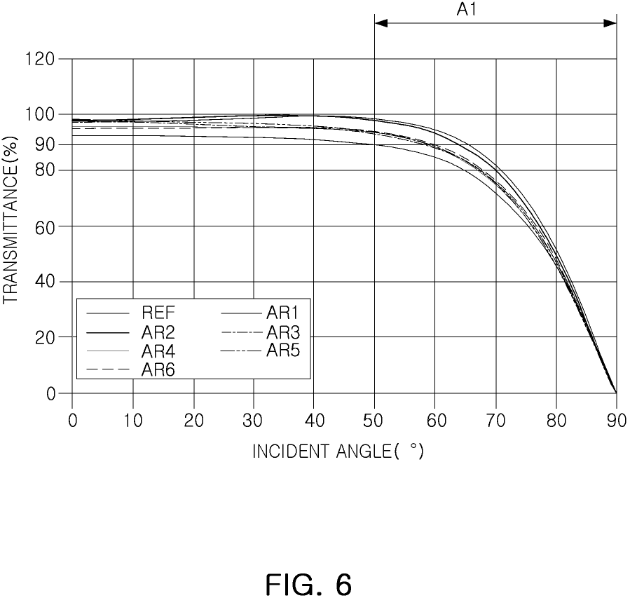

The transmittance and a maximum transmittance angle of the antireflective layer 420 will be described with reference to FIGS. 5 and 6. FIG. 5 is a graph illustrating transmittance and a maximum transmission angle according to the type of antireflective layer in the semiconductor ultraviolet light emitting device packages of an Example and a Comparative Example, and FIG. 6 is a graph illustrating a relationship between an incident angle of ultraviolet light incident on the antireflective layers formed in each of the semiconductor ultraviolet light emitting device packages of an Example and a Comparative Example, and transmittance.

FIG. 5 shows a graph measuring a transmittance and a maximum transmission angle of a Comparative Example in which an antireflective layer 420 is not formed in the semiconductor ultraviolet light emitting device package, and a transmittance and a maximum transmission angle of semiconductor ultraviolet light emitting device package in which various types of the antireflective layer 420. In a first Example AR1, a multilayer film of MgF₂/Al₂O₃ is formed on both the upper surface 410U and the lower surface 410B of the light transmitting substrate 410. In a second Example AR2, a multilayer film of MgF₂/HfO₂ is formed on both the upper surface 410U and the lower surface 410B of the light transmitting substrate 410. In a third Example AR3, a multilayer film of Al₂O₃/MgF₂ is formed only on the upper surface 410U of the light transmitting substrate 410. In a fourth Example AR4, a multilayer film of Al₂O₃/SiO₂ is formed only on the upper surface 410U of the light transmitting substrate 410. In a fifth Example AR5, a multilayer film of MgF₂/Al₂O₃/SiO₂ is formed only on the upper surface 410U of the light transmitting substrate 410. In a sixth Example AR6, a multilayer film of Al₂O₃/MgF₂/Al₂O₃/SiO₂ is formed only on the upper surface 410U of the light transmitting substrate 410.

In the case of a Comparative Example (REF), it can be seen that vertical transmittance, which is transmittance when ultraviolet light are vertically incident, is about 92%, and a maximum transmittance angle is about 50°. On the other hand, in the case of the first to sixth Examples, it can be seen that a vertical transmittance is about 95% or more, and a maximum transmission angle is about 57° or more. Accordingly, it can be seen that vertical transmittance and maximum transmittance angle are increased in the first to sixth Examples compared to Comparative Examples. In addition, it can be seen that the transmittance when the ultraviolet ray is obliquely incident is also increased from an increase in the maximum transmission angle. For example, it can be seen that when ultraviolet light is incident obliquely,in the case of Comparative Example (REF), the transmittance becomes 90% or more only within 0° to about 50°, but in the case of Examples 1 to 6, a range having a transmittance of 90% or more is increased to 65°.

Referring to FIG. 6, it can be seen that, in both the Comparative Example REF and the first to sixth Examples AR1 to AR6, the transmittance tends to decrease as an incident angle increases in a region A1 in which the incident angle is 50° or more, but in the first to sixth Examples AR1 to AR6, an amount of decrease in transmittance is lower than that of the Comparative Example REF. In addition, in the case of the Comparative Example REF, when the incident angle exceeds 50°, it can be seen that the transmittance is reduced to less than 90%. That is, it can be seen that the maximum transmission angle is only 50° in the Comparative Example REF. On the other hand, in the first to sixth Examples AR1 to AR6, it can be seen that the transmittance is reduced to be less than 90% when the incident angle exceeds at least 55°. That is, it can be seen that the maximum transmission angle is increased to 55° or more in the first to sixth Examples AR1 to AR6. Accordingly, it can be seen that a maximum transmission angle in the first to sixth examples AR1 to AR6 is increased compared to that of in the Comparative Example REF, and an irradiation angle of light is decreased. In the case of the first to sixth example examples (AR1 to AR6), it was measured that light is irradiated at an irradiation angle of less than 100°.

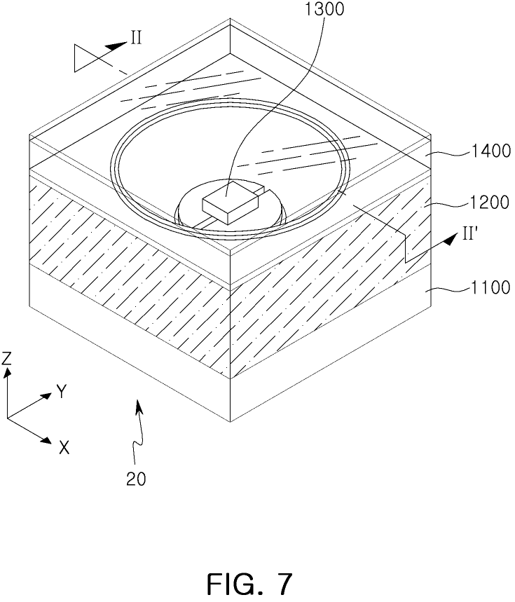

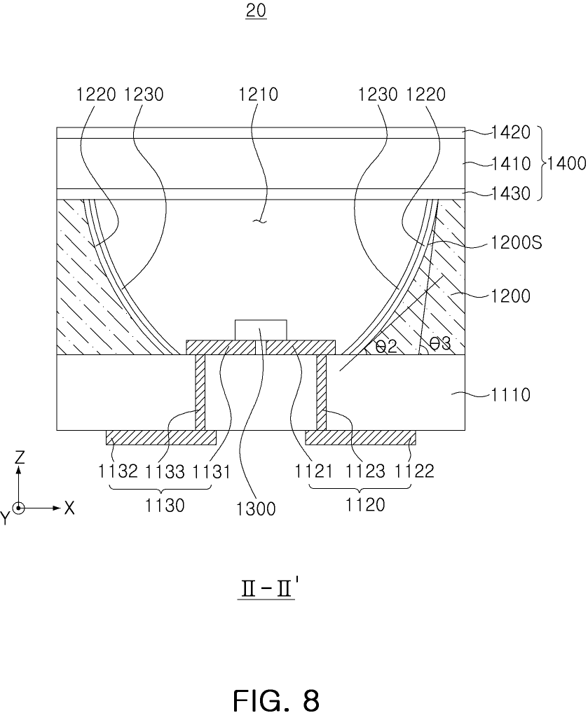

Referring to FIGS. 7 and 8, a semiconductor ultraviolet light emitting device package 20 according to an example embodiment will be described. FIG. 7 is a perspective view of a semiconductor UV light emitting device package according to an example embodiment of the present disclosure, and FIG. 8 is a cross-sectional view of the semiconductor UV light emitting device package of FIG. 1 taken along line II-II'.

Compared to the semiconductor ultraviolet light emitting device package 10 described above, a semiconductor ultraviolet light emitting device package 20 according to an example embodiment has a difference in that a reflector 1200 has a sidewall 1200S that is curved. In addition, there is a difference in that a first antireflective layer 1420 and a second antireflective layer 1430 are respectively formed on upper and lower surfaces of a light transmitting cover 1400. Other configurations are the same as those of the semiconductor ultraviolet light emitting device package 10 of FIG. 1 described above, and thus a description thereof will be omitted.

In the reflector 1200 of an example embodiment, the sidewall 1200S has a curved surface, and a slope of the curved surface may gradually increase from the upper surface 100U to the light transmitting cover 1400. That is, the slope of the curved surface may gradually increase as it moves away from the semiconductor ultraviolet light emitting device 1300. In an example embodiment, a first slope θ2 in contact with the package substrate 1110 may have an angle, exceeding 55°, and a second slope θ3 may have an angle, less than 75°.

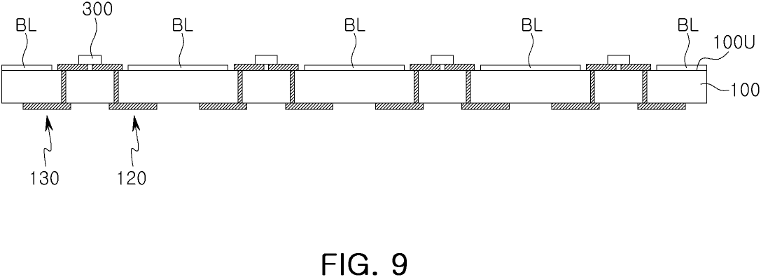

Next, a manufacturing process of the semiconductor ultraviolet light emitting device package will be described with reference to FIGS. 9 to 12. FIGS. 9 to 12 are cross-sectional views schematically illustrating a manufacturing process of the semiconductor ultraviolet light emitting device package of FIGS. 1 and 2.

Referring to FIG. 9, a plurality of the semiconductor ultraviolet light emitting device 300 may be mounted on an upper surface 100U of a package substrate 100 on which the first electrode structure 120 and the second electrode structure 130 are disposed, and an adhesive layer BL may be applied between the plurality of the semiconductor ultraviolet light emitting device 300. The adhesive layer BL may be formed of a material such as a silicone resin, an epoxy resin, an acrylic resin, or silicone. However, in some example embodiments, the adhesive layer BL may be omitted.

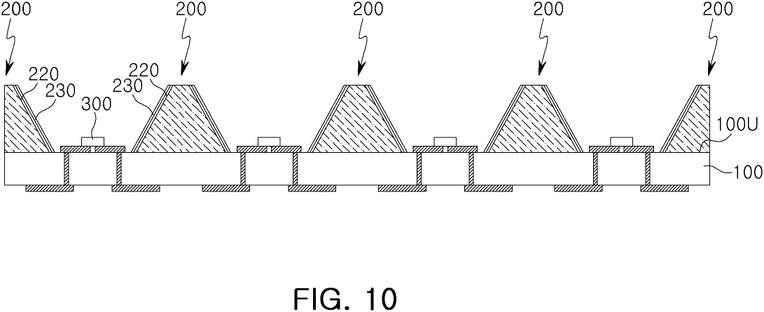

Next, referring to FIG. 10, a reflector 200 may be attached to the upper surface 100U of the package substrate 100. In some example embodiments, before the reflector 200 is attached, a reflective layer 220 and a protective layer 230 may be first formed on a sidewall 200S of the reflector 200. The reflector 200 may be formed by forming a body of ceramic or silicon having relatively low reflectivity, and covering the sidewall 200S with a reflective layer 220 formed of a material having high reflectivity such as aluminum. The protective layer 230 may be formed by depositing any one or a combination of SiO₂, Al₂O₃, AlN, and Si₃N₄ on a surface of the reflective layer 220.

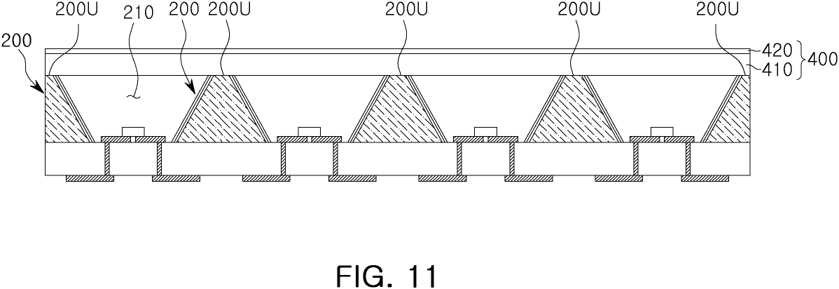

Next, referring to FIG. 11, a light transmitting cover 400 may be attached to an upper surface of the reflector 200 to cover an opening 210. The light transmitting cover 400 may be attached to the upper surface 200U of the reflector 200 by an adhesive layer. The adhesive layer may be formed of a material such as a silicone resin, an epoxy resin, an acrylic resin, a metal layer, and water glass or silicone. However, depending on the example embodiment, the adhesive layer may be omitted, and a light transmitting cover 400 may be attached to the upper surface 200U of the reflector 200 by methods such as anodic bonding, fusion bonding, eutectic bonding, welding, or the like.

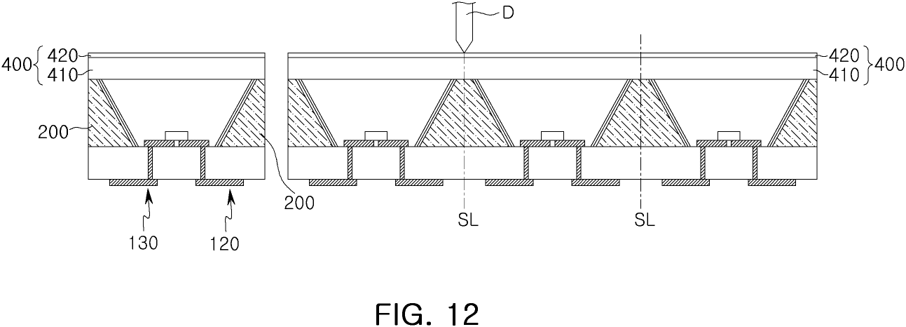

Next, referring to FIG. 12, when a semiconductor ultraviolet light emitting device is cut into units of individual devices along a cutting line SL using a saw D, the semiconductor ultraviolet light emitting device package 10 shown in FIG. 2 may be manufactured.

As set forth above, according to an aspect of the present disclosure, in a semiconductor ultraviolet light emitting device package, ultraviolet light having a narrow orientation angle can be emitted, by optimizing an angle of an inclination angle of a reflector disposed around the semiconductor ultraviolet light emitting device and transmittance of an antireflective layer of a light transmitting cover through which the emitted UV light is transmitted.

Various and advantageous advantages and effects of embodiments of the present disclosure are not limited to the above description, and will be more readily understood in the process of describing the specific embodiments of the present disclosure.

While non-limiting example embodiments have been shown and described above, it will be apparent to those skilled in the art that modifications and variations could be made without departing from the scope of the present disclosure.

1. A semiconductor ultraviolet light emitting device package, comprising:
a package substrate comprising a first surface and a second surface, opposite to the first surface;
a semiconductor ultraviolet light emitting device mounted on the first surface of the package substrate and configured to emit deep ultraviolet light including a wavelength in a range of 250 nm to 285 nm;
a reflector disposed on the first surface of the package substrate to surround the semiconductor ultraviolet light emitting device, and comprising an inclined sidewall that defines an opening of the reflector, the semiconductor ultraviolet light emitting device disposed within the opening; and
a light transmitting cover comprising a lower surface covering the opening and an upper surface opposite to the lower surface, wherein an antireflective layer is disposed on at least one from among the lower surface and the upper surface,
wherein the inclined sidewall has an inclination angle greater than 55° and less than 75° with respect to the first surface,
wherein a distance between an upper surface of the semiconductor ultraviolet light emitting device and the lower surface of the light transmitting cover is 100 µm to 2000 µm, and
wherein a refractive index of the antireflective layer is in a range of 1.3 to 2.5.

2. The semiconductor ultraviolet light emitting device package of claim 1, wherein the reflector further comprises
a reflector body in which the opening is disposed, the reflector body comprising the inclined sidewall; and
a reflective layer disposed on the inclined sidewall that defines the opening.

3. The semiconductor ultraviolet light emitting device package of claim 2, wherein the reflector body is formed of a material comprising at least one from among ceramic, metal, and silicon,
wherein the reflective layer is formed of a material comprising aluminum (Al).

4. The semiconductor ultraviolet light emitting device package of claim 1, wherein the reflector is formed of a material comprising aluminum (Al).

5. The semiconductor ultraviolet light emitting device package of claim 1, further comprising a protective layer covering the inclined sidewall that defines the opening,
wherein the protective layer is formed of a material comprising at least one from among SiO₂, Al₂O₃, A1N, and Si₃N₄.

6. The semiconductor ultraviolet light emitting device package of claim 5, wherein a thickness of the protective layer is in a range of 5 nm to 50 nm.

7. The semiconductor ultraviolet light emitting device package of claim 1, wherein the antireflective layer comprises at least one first refractive layer, having a first refractive index, and at least one second refractive layer, having a second refractive index that is higher than the first refractive index, wherein the at least one first refractive layer and the at least one second refractive layer are alternately stacked.

8. The semiconductor ultraviolet light emitting device package of claim 1, wherein the antireflective layer comprises at least one from among MgF₂, SiO₂, Al₂O₃, and HfO₂.

9. The semiconductor ultraviolet light emitting device package of claim 1, wherein the package substrate is formed of a material comprising ceramic.

10. The semiconductor ultraviolet light emitting device package of claim 1, wherein the sidewall that defines the opening is planar.

11. The semiconductor ultraviolet light emitting device package of claim 1, wherein the sidewall that defines the opening comprises a curved surface, and a slope of the curved surface gradually increases from the first surface of the package substrate to the light transmitting cover.

12. The semiconductor ultraviolet light emitting device package of claim 1, wherein the opening has a height of 300 µm to 2200 µm with respect to the first surface of the package substrate.

13. A semiconductor ultraviolet light emitting device package, comprising:
a body having a cavity;
a semiconductor ultraviolet light emitting device mounted on a surface of the body that defines a bottom of the cavity;
a light transmitting cover comprising a light transmitting substrate covering the cavity; and
an antireflective layer disposed on at least one from among an upper surface and a lower surface of the light transmitting substrate,
wherein the body comprises a sidewall that defines sides of the cavity and has an inclination angle that is greater than 55° and less than 75° with respect to the surface of the body that defines the bottom of the cavity,
wherein a distance between an upper surface of the semiconductor ultraviolet light emitting device and a lower surface of the light transmitting cover is 100 µm to 2000 µm, and
wherein a refractive index of the antireflective layer is in a range of 1.3 to 2.5.

14. The semiconductor ultraviolet light emitting device package of claim 13, wherein the body comprises:
a package substrate that comprises the surface that defines the bottom of the cavity; and
a reflector disposed on the package substrate and comprising the inclined sidewall that defines the sides of the cavity,
wherein the reflector is formed of a material comprising at least one from among ceramic, metal, and silicon, and
wherein a reflective layer comprising aluminum (Al) is disposed on the sidewall of the reflector.

15. The semiconductor ultraviolet light emitting device package of claim 14, further comprising
a protective layer covering the sidewall of the reflector,
wherein the protective layer is a formed of a material comprising at least one from among SiO₂, Al₂O₃, AlN, and Si₃N₄.

16. The semiconductor ultraviolet light emitting device package of claim 13, wherein the semiconductor ultraviolet light emitting device is configured to emit deep ultraviolet light including a wavelength in a range of 250 nm to 285 nm.

17. The semiconductor ultraviolet light emitting device package of claim 16, wherein the semiconductor ultraviolet light emitting device is configured to emit light of a first orientation angle,
wherein the light of the first orientation angle emitted from the semiconductor ultraviolet light emitting device passes through the light transmitting cover and is refracted at a second orientation angle, narrower than the first orientation angle,
wherein the second orientation angle is an angle that is less than 100°.

18. The semiconductor ultraviolet light emitting device package of claim 13, wherein the light transmitting substrate comprises at least one from among soft glass, fused silica, and fused quartz.

19. A semiconductor ultraviolet light emitting device package, comprising:
a package substrate;
a reflector disposed on the package substrate and comprising an inclined sidewall that defines an opening of the reflector;
a semiconductor ultraviolet light emitting device mounted on the package substrate and disposed in the opening; and
a light transmitting cover comprising a lower surface covering the opening and an upper surface opposite to the lower surface, wherein an antireflective layer is disposed on at least one from among the lower surface and the upper surface,
wherein the inclined sidewall has an inclination angle that is greater than 55° and less than 75° with respect to an upper surface of the package substrate, and
wherein a refractive index of the antireflective layer is in a range of 1.3 to 2.5.

20. The semiconductor ultraviolet light emitting device package of claim 19, wherein a thickness of the antireflective layer is in a range of 25 nm to 400 nm.

21. (canceled)
